(19) United States
(12) Patent Application Publication
(10) Pub. No.: US 2016/0206388 A1
(43) Pub. Date: Jul. 21, 2016

(54) FLEXIBLE SURGICAL INSTRUMENT WITH LINKS UNDERGOING SOLID-STATE TRANSITIONS

(71) Applicant: Intuitive Surgical Operations, Inc., Sunnyvale, CA (US)

(72) Inventor: Giuseppe Maria Prisco, Calci (IT)

(21) Appl. No.: 15/079,411

(22) Filed: Mar. 24, 2016

Related U.S. Application Data
(62) Division of application No. 12/343,274, filed on Dec. 23, 2008, now abandoned.

(51) Int. Cl. A61B 34/30; A61B 34/35

(52) U.S. Cl. CPC A61B 34/30; A61B 34/35; A61B 2034/301

(57) ABSTRACT

A surgical instrument has a tip section with several degrees of freedom of articulation and at least one link that may be too long for insertion through an entry guide that follows a curved path. Each long link is made of a shape memory alloy or another material having a state in which the link is sufficiently flexible to bend as needed to pass through the entry guide. Once through the entry guide, the material of the link makes a transition to a state in which the link returns to a desired shape and is sufficiently rigid for precise controlled movement against external forces and for actuation using tendons.

### BACKGROUND

Minimally invasive surgical procedures allow diagnostic tests and corrective surgeries with a minimal amount of damage to healthy tissues. For example, laparoscopic surgery, which is minimally invasive surgery on the abdomen, generally introduces multiple surgical instruments through small incisions in a patient. The inserted instruments typically have small diameter rigid extensions or main tubes with end effectors that can be manually or robotically controlled to perform a desired surgical procedure. Laparoscopic surgery typically uses two or more incisions to provide separation between the instruments and to allow insertion of the instruments from different directions for triangulation on a work site inside the body. The separation and triangulation of instruments is often critical to allowing the instruments to work cooperatively during surgical manipulations.

A single port minimally invasive procedure can be performed using a single small incision through which all needed instruments are inserted. The use of a single incision may allow single port systems to perform surgical procedures with even less damage to healthy tissue. However, with a single port system, separation and triangulation of working instruments is more difficult to achieve since all of the instruments are inserted along the same direction and path. U.S. Pat. App. Pub. No. US 2008/0065105, entitled “Minimally Invasive Surgical System,” of Larkin et al. discloses some single port minimally invasive surgical systems and is hereby incorporated by reference in its entirety. FIG. 1 shows the distal end of a single-port surgical system 100 disclosed by Larkin et al. System 100 includes two tools or end effectors 110 and 120 and a camera system 130 that are all inserted through an entry guide 140. To achieve separation, end effectors 110 and 120 are at the ends of respective wrist mechanisms including joints with relatively long links 112 and 122, respectively. The long links 112 and 122 can remain parallel to a straight entry guide 140 during insertion for a surgical procedure. Once inserted past entry guide 140, small rotations of links 112 and 122 about respective proximal joints 114 and 124 create relatively large separations between end effectors 110 and 120 and permit triangulation of end effectors 110 and 120 on the work site.

Minimally invasive surgical instruments are being developed that have flexible main tubes that are able to bend as needed to follow a natural lumen, such as a portion of the digestive tract of a patient, or for insertion through an entry guide that bends as needed to follow a natural lumen in the patient. Whether inserted directly or through an entry guide, these flexible medical instruments will generally need to make several bends at locations that will vary during a procedure and vary from one procedure to the next. Accordingly, these flexible instruments cannot employ long, rigid links that are unable to navigate the curves required to reach the work site. As a result, without long rigid links, flexible instruments inserted through the same entry guide often have little separation from one another and little or no triangulation relative to each other. This makes basic surgical manipulations such as suturing difficult, if not impossible to accomplish with conventional flexible medical instruments. In view of this problem, it would be desirable to have simple devices and procedures for achieving useful triangulation and working separation between instruments at the distal end of a flexible instrument.

### SUMMARY

In accordance with an aspect of the invention, a flexible surgical instrument has a distal tip section with several degrees of freedom of articulation and at least one link that may be too long for insertion through an entry guide that follows a natural lumen inside a patient. However, each long link contains a shape memory alloy or another material that can make a transition to a state in which the link is sufficiently flexible to pass through bends in the entry guide. Once through the entry guide, the material of the link makes a transition to a state in which the link returns to its original shape and is sufficiently rigid for precise controlled movement against external forces and for actuation using tendons.

One specific embodiment of the invention is a surgical system including a main tube, a tip section, a tendon, and a temperature control system. The tip section is at a distal end of the main tube and includes a link containing a material, such a shape memory alloy, that can reversibly transform between a first state and a second state. The tendon extends through the main tube and is coupled to the link so that movement of the tendon can cause actuation of the link about a joint in the tip section. The temperature control system operates to change the temperature of the link to cause transitions between the first temperature and the second temperature. In the first state, the material is flexible enough to permit bending of the link during insertion of the instrument though a bent entry guide. In the second state, the material is stiffer and permits the tendon to actuate the link against external forces during a surgical procedure.

Another specific embodiment of the invention is a surgical process. The process includes inserting an entry guide in a patient and inserting a tip section of an instrument through the entry guide. During insertion of the tip section, a material in a link in the tip section is kept in a first state that provides the link with sufficient flexibility to bend while being inserted. Once the tip section has been inserted through the entry guide, the process changes a temperature of the link to cause the material in the link to transition to a second state, in which the material is more rigid than the material is in the first state. While the material is in the second state, the link can be actuated using a tendon.

### BRIEF DESCRIPTION OF THE DRAWINGS

FIG. 1 shows a known single port system using long links to achieve a large working volume and separation and triangulation of end effectors.

FIG. 2 is a plot illustrating heating and cooling curves for the martensitic transitions of a material used in a link of a flexible instrument in accordance with an embodiment of the invention.

FIG. 3A shows a surgical instrument in accordance with an embodiment of the invention using a link that undergoes a solid-state transition between insertion along a curved path and use at a work site.

FIG. 3B shows a more detailed view of a tip section of the surgical instrument of FIG. 3A.

FIG. 4 shows a link of a surgical instrument in accordance with an embodiment of the invention employing electrical resistive heating to cause a solid-state transition in the link.

FIG. 5 shows a link of a surgical instrument in accordance with an embodiment of the invention employing a fluid path for a heated or cooled liquid that causes a solid-state transition in the link.

FIG. 6 shows a surgical system in accordance with an embodiment of the invention employing robotic control.

Use of the same reference symbols in different figures indicates similar or identical items.

### DETAILED DESCRIPTION

In accordance with an aspect of the invention, a surgical system includes a flexible endoscope or entry guide that can be inserted in the body of a patient and steered to desired surgical links. One or more surgical instruments, each of which may be robotically controlled, can then be deployed via available lumens in the entry guide. (As used herein, the terms “robot” or “robotically” and the like include teleoperation or telerobotic aspects.) The surgical instruments have a flexible main tube and a distal tip section with several degrees of freedom of articulation to provide enough dexterity so that a surgeon using the instruments can effectively perform complex surgical tasks, such as cutting and suturing. One effective kinematic embodiment of a surgical instrument has a tip section with a long link to provide more work volume for the instrument tip. The links in the instrument tip need to be rigid during surgery so that an exact kinematic control of tip movement can be achieved in the presence of external forces. However, the entry guide may take up a tightly bent shape to follow a path through a natural orifice and a natural lumen of the patient's body, and an instrument with long, rigid links may not be able to navigate the bends in the entry guide. In accordance with an aspect of the invention, a long link in a surgical instrument is made of a shape memory alloy or another material that can make a solid-state transition to a state in which the long link is sufficiently flexible to pass through bends in the entry guide. Once through the entry guide, the material of the link makes a solid-state transition to a state in which the link is sufficiently rigid for precise controlled movement against external forces.

One class of material suitable for a link having both a flexible state and a rigid state is a shape memory alloy or other material that can undergo a martensitic transition (i.e., a transition between a martensite state having a martensite crystal structure and an austenite state having an austenite crystal structure) when the temperature of the material changes. The temperature change required to produce the austenite to martensite transition generally has thermal hysteresis curves such as illustrated in FIG. 2. As illustrated in FIG. 2, the material at low temperatures is in a martensite state but when heated to a temperature Aₛ (austenite start) begins to transition to an austenite state. The transition to the austenite state occurs over a temperature range from temperature Aₛ to a temperature A_{f} (austenite finish), and above temperature A_{f} the material is about one-hundred percent (100%) in the austenite state. If the material is cooled from a temperature above temperature A_{f}, the material transitions back to the martensite state as the material drops from a temperature Mₛ (martensite start) to a temperature M_{f} (martensite finish).

The temperatures Aₛ, A_{f}, Mₛ, and M_{f} associated with the martensitic transition depend on the material and may also depend on the stress applied to the material. For binary nickel-titanium (NiTi) alloys, the transformation temperature hysteresis, which is generally defined as the difference between the temperatures at which the material is 50% transformed to austenite upon heating and 50% transformed to martensite upon cooling, is typically about 25 to 50° C. However, alloy additions can be used to manipulate the thermal hysteresis. For example, the addition of copper (Cu) to a NiTi alloy can reduce the width of the transformation temperature hysteresis to about 10° C. to 15° C.

The material of the link may have a Young's modulus in the martensite state that is several times lower than the Young's modulus of the material in the austenitic state. As a result, the link when fully or partly in the martensite (or low temperature) state can be sufficiently flexible to bend as needed for insertion through a curved guide to a work site in a patient. Heating of the link at the work site can cause the link to transition to the austenite state, which increases the stiffness of the link and causes the shape of the link to return back to its original shape, regardless of the shape that the link was bent into when cold. The link during use will thus be rigid and have a shape suitable for precise movement of the working surfaces of the instrument.

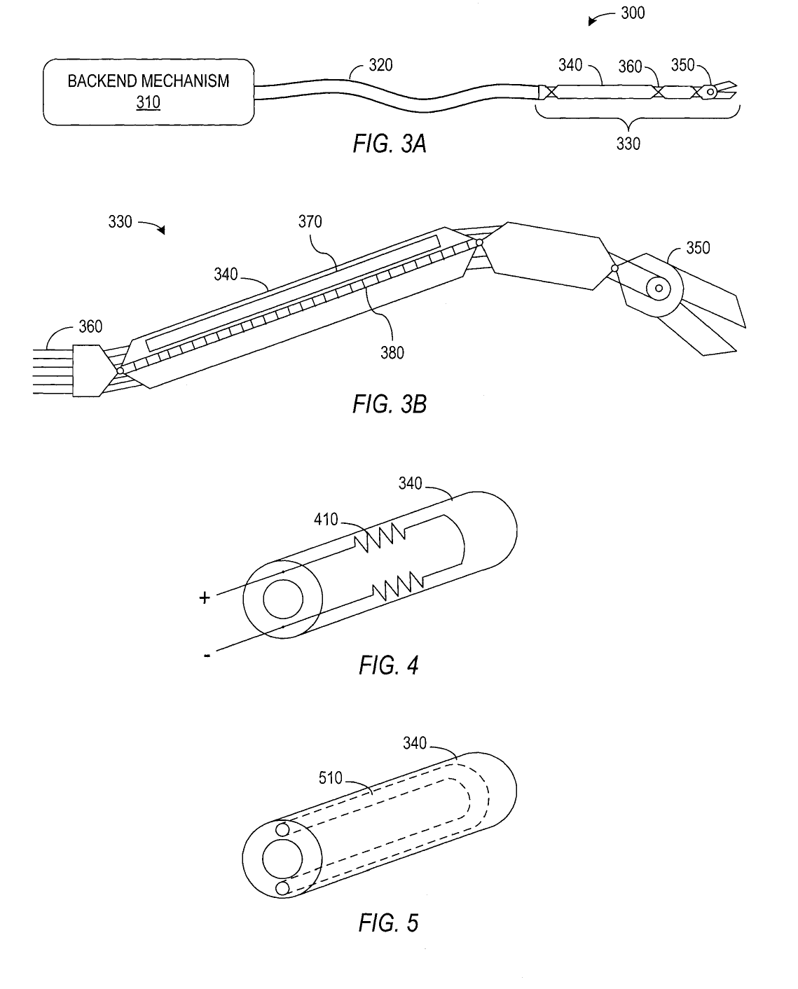

FIG. 3A shows a surgical instrument 300 in accordance with an embodiment of the invention. Instrument 300 includes a backend mechanism 310, a flexible main tube 320, and a tip section 330. Tip section 330 includes one or more links 340 and an end effector 350 that are articulated using tendons 360. Tendons 360 may be cables, tubes, or similar structures that extend back through flexible main tube 320 to backend mechanism 310. For robotic control of instrument 300, backend mechanism 310 contains a transmission with a mechanical interface adapted for connection to a motor package (not shown), and through backend mechanism 310, tendons 360 are connected to motors that can pull on tendons 360 to actuate tip section 330.

Tip section 330 can generally employ any desired mechanical structure that provides tip section 330 with actuated degrees of freedom of motion that are needed or desirable for performing a surgical operation. In the illustrated embodiment, end effector 350 of tip section 330 has jaws that can rotate about a pivot and are connected to corresponding tendons 360 so that backend mechanism 310 pulling on the correct tendon can causes a jaw to rotate clockwise or counterclockwise about the pivot. The jaws of end effector 350 in the illustrated embodiment can be forceps or scissors that are used to perform functions such as gripping or cutting, but many types of end effectors are known in the art and could be employed in alternative embodiments of tip section 330. Tip section 330 also includes links with joints, and specific tendons 360 connected to the links so that backend mechanism 310 pulling on the correct tendon can cause a link to rotate about a joint on the proximal end of the link. Many mechanical systems for the tip sections of surgical instruments are known and could be employed in tip section 330. In particular, U.S. Pat. App. Pub. No. 2008/0065105, entitled “Minimally Invasive Surgical System,” of Larkin et al., which is incorporated by reference above, describes in more detail some examples of suitable mechanical structures for tip section 330.

One characteristic of tip section 330 is that tip section 330 includes at least one link 340 that provides tip section 330 with a desired working volume or range but may be too long for insertion through an entry guide without bending of link 340. In accordance with an aspect of the invention, link 340, which is shown in more detail in FIG. 3B, is made of a material such as a shape memory alloy having a martensite state, which is more flexible and has a low Young's modulus and/or high ductility and malleability, and an austenite state, which is more rigid and has a higher Young's modulus and/or lower ductility and malleability. A material with martensite state that is ductile and malleable may be desirable, so that bending of link 340 during insertion through a guide causes mostly inelastic or plastic deformations. Otherwise, if the deformation of link 340 is elastic, the energy stored in the deformation of link 340 will be released when link 340 pass out of the guide, and the energy release can create undesirable movement or vibration at tip 330. In one exemplary embodiment, link 340 has a body that is a tube of Nitinol alloy with hysteresis temperatures Mₛ, M_{f}, Aₛ, and A_{f} that are about 24° C., 36° C., 54° C., and 71° C., a martensite state with a Young's modulus of about 4×10⁶ to 6×10⁶ psi, and an austenite state with a Young's modulus of about 12×10⁶ psi. The characteristics of Nitinol alloys generally depend on their composition, and significant freedom is available to design an alloy having a desirable thermal hysteresis for martensite transition and flexibility in the martensite state. Some other suitable materials for link 340 include but are not limited to NiTi, CuAlNi, CuAl, CuZnAl, TiV, and TiNb. Generally, link 340 will be kept in the more flexible (e.g., martensite) state during insertion of instrument and will only be actuated when link is in the stiffer (e.g., austenite) state. The preloaded tensions in tendons 360 can be kept low to avoid buckling of link 340 when link 340 is in the flexible or martensite state, particularly when link 340 is not supported by an entry guide.

Link 340 also includes a heating system 370 and a shape sensor 380 as shown in FIG. 3B. Heating system 370 can be a resistor or other electrically resistive structure 410, which may be embedded in the walls of link 340 as shown in FIG. 4. Heating system 370 can be connected to wires that extend back through flexible main tube 320 or in the walls of flexible main tube 320 to an electrical interface associated with backend mechanism 310. Once link 340 has reached a work site for a surgical procedure, a control system (not shown) driving a current through resistive element 410 can heat link 340 to a temperature high enough to cause link 340 to transition from the more flexible martensite state to the stiffer austenite state.

FIG. 5 illustrates an embodiment of link 340 containing a fluid path or pipe 510 in the walls of link 340. Pipe 510 may be used for cooling of link 340. The ends of pipe 510 may be connected to a source pipe and a drain pipe that run through flexible main tube 320 to backend mechanism 310, and the source pipe may be connected to a fluid source such as a water pump that circulates cool water through pipe 510. Alternatively, cooling may be achieved without pipe 510 in the walls of link 340 simply by running water or other cool liquid through link 340 and using a separate suction or return path to remove liquid.

Pipe 510 can more generally change the temperature of link 340 according to the temperature of the liquid circulated. In particular, cool water can reduce the temperature of link 340. Alternatively, hot water can be run through pipe 510 to heat link 340 with or without the assistance of electrical resistive heating. Heating of link 340 serves to cause the transition of link 340 to the austenite state as described above. Cooling of link 340 is optional but may be desirable to speed up the transition from the austenite state of link 340 even when the environment surrounding link 340 is cooler than final martensite transition temperature M_{f} for the body material of link 340. Alternatively, the body material of link 340 may have a final austenite transition temperature A_{f} that is lower than the temperature of the surrounding environment, in which case cooling is required to achieve the transition of link 340 to the martensite state. In general, it is desirable to have the final martensite transition temperature M_{f} or at least the start martensite temperature Mₛ higher than the temperature of the surrounding environment so that link 340 will be flexible and therefore can still be removed in the event of a malfunction of cooling system 510. The final austenite transition temperature A_{f} may be about 10° C. or more higher than the body temperature of the patient.

Shape sensor 380 as shown in FIG. 3B can be implemented using a fiber Bragg grating sensor such as described in U.S. patent application Ser. No. 12/164,829, entitled “Fiber Optic Shape Sensor,” by Giuseppe M. Prisco, which is hereby incorporated by reference in its entirety. Shape sensor 380, which may extend back through flexible tube 320 to backend mechanism 310, can be used to determine the exact shape/orientation of flexible main tube 320, link 340, and other portions of tip section 330 relative to backend mechanism 310. Shape sensing may be desirable particularly when link 340 returns to the austenitic state after being bent, since even a shape memory alloy may not return exactly to the shape associated with the austenite state. A robotic control system can take the measured shape of link 340 into account for a kinematically exact control of tip section 330 through manipulation of tendons 360.

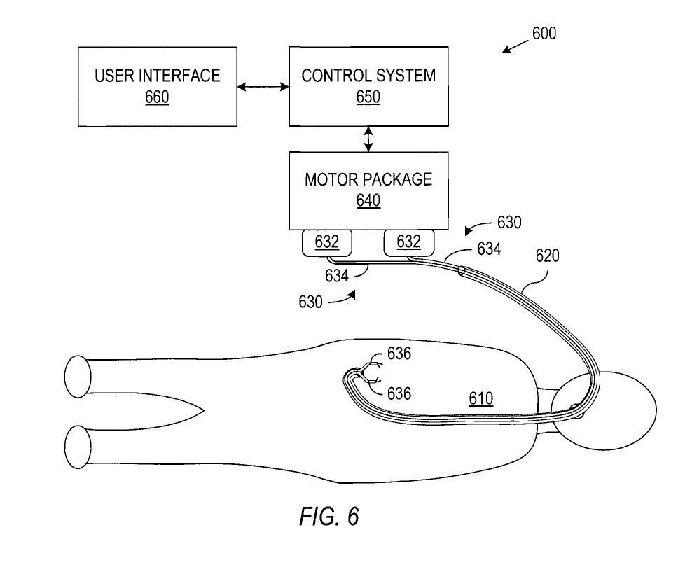

FIG. 6 illustrates a system 600 for performing a minimally invasive surgical procedure on a patient 610. System 600 employs a flexible entry guide 620 that can be inserted though a natural orifice such as the mouth of patient 610 and directed along a natural lumen such as the digestive tract of patient 610. One or more flexible instruments 630 and a vision system (not shown) can be inserted through entry guide 620. FIG. 6 shows an example in which two instruments 630 are inserted though separate lumens in entry guide 620. Alternatively, one instrument or three or more instruments could be inserted through entry guide 620 so that tip sections of the instruments are at a work site in patient 610.

Each instrument 630 includes a backend mechanism 632, a flexible main tube 634, and a tip section 636 that may be substantially identical to backend mechanism 310, flexible main tube 320, and tip section 330, which are described above with reference to FIGS. 3A and 3B. In particular, each tip sections 636 of instruments 630 may contain links that are too long to be inserted through a tight fitting lumen in entry guide 620 without bending the link. For the insertion of an instrument 630 through entry guide 620, long links contain a shape memory alloy that is kept at a temperature in which the bodies of the links are in a more flexible martensite state. The temperature environment (e.g., room temperature or the body temperature of patient 610) is preferably below hysteresis temperature Mₛ so that no cooling is needed to keep the links in the martensite state. Accordingly, the links in tip section 636 can be bent during insertion as needed to slide the long links around turns in entry guide 620. Using a material with temperature Mₛ above the temperature of the environment can improve the safety of instruments 630, in that if a warming or cooling system for an instrument 630 fails, the links in instrument 630 return to a relatively flexible state that allows instruments 630 to be withdrawn from patient 610.

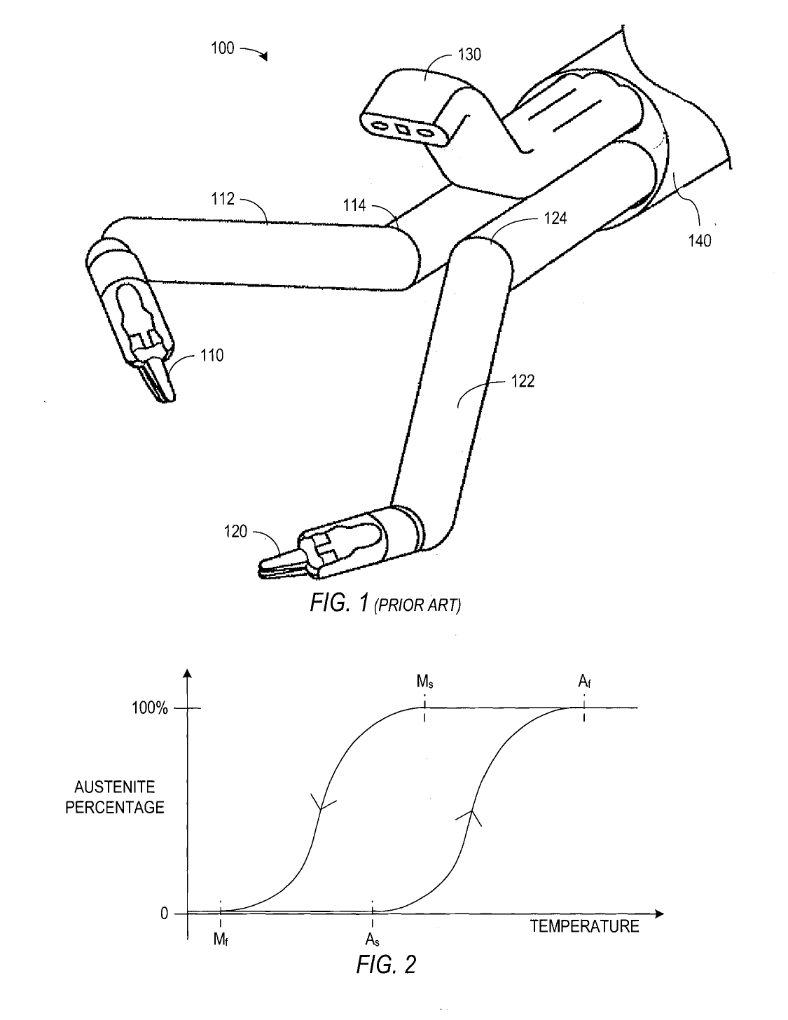

Tip sections 636 emerge from the distal end of guide tube 620 at the end of the insertion process for instruments 630. Each tip section 636 is then at a work site in patient 610. For actuation using tendons as described above, the links in the martensite state are heated to cause a transition to the austenite state. The transition to the austenite state causes the links to straighten or otherwise return to a shape associated with the austenite state and also become stiffer, so that the links are able to withstand the applied forces and torques during actuation using the tendons extending to backend mechanism 632. The long links of each instrument 630 provide a large working volume or range of motion for each tip section 636, which can improve the versatility and functionality of the instrument 630. In particular, with two instruments 630 as shown in FIG. 6, long links in tip sections 636 permit large separation of end effectors in the tip sections 636 and permit triangulation of the end effectors for surgical tasks, such as suturing. Instruments 630 can thus achieve the same functionality of a known single port systems such as system 100 of FIG. 1 and do so at the distal end of an entry guide 620 that follows a path with bends that are too sharp for insertion of straight rigid links used in the known system.

Tendons, which can be used for control of the tip section 636, run through flexible main tube 634 to backend mechanism. Backend mechanisms 632 connect to a motor package 640 that contains motors that drive backend mechanisms 632 to control tensions in the tendons as required for operation of instruments 630. An interface for sensor signals (e.g., from shape sensors) and video signals from a vision system inserted through guide tube 620 may be provided through package 640, a control system 650, or a user interface 660. Electrical or other power and communication signals could also be sent to or received from sensors or control electronics in tip sections 636. User interface 660 preferably provides an operator, e.g., a surgeon, with a visual display, such as a stereoscopic (3-D) display, and includes manipulator controls that the operator moves to operate tip sections 636. Control system 650 can use measurements of the shapes of links in tip sections 636 in conversions of the surgeon's movements of the manipulators in user interface 660 into control signals that cause motor package 640 to apply tension to drive tendons as needed to provide the desired movement of tip sections 636. Some suitable user interfaces and control systems for endoscopic surgical systems are further described in U.S. Pat. No. 5,808,665, entitled “Endoscopic Surgical Instrument and Method for Use,” to Philip S. Green; which is hereby incorporated by reference in its entirety.

Although the invention has been described with reference to particular embodiments, the description is only an example of the invention's application and should not be taken as a limitation. Various adaptations and combinations of features of the embodiments disclosed are within the scope of the invention as defined by the following claims.

What is claimed is:

1. A surgical system comprising:
a main tube;
a tip section at a distal end of the main tube, the tip section including a link comprising a material that transforms from a first state to a second state upon heating to a first temperature and transforms from the second state to the first state upon cooling from the first temperature to a second temperature;
a tendon extending through the main tube and coupled to the link, wherein movement of the tendon causes actuation of the link about a joint in the tip section; and
a temperature control system for changing a temperature of the link.

2. The system of claim 1, further comprising a guide that is sufficiently flexible to bend as needed to follow a curved path inside a patient, wherein the link is kept in the first state to allow insertion of the link through a lumen of the guide and kept in the second state for actuation using the tendon.

3. The system of claim 1, wherein the first state is a martensite state, and the second state is an austenite state.

4. The system of claim 1, wherein transition of the material from the first state to the second state stiffens the link for performing surgery with the surgical system.

5. The system of claim 1, wherein transition of the material from the first state to the second state removes bends made in the link during insertion of the instrument for a surgical procedure.

6. The system of claim 1, wherein the link comprises a shape memory alloy.

7. The system of claim 1, wherein the temperature control system comprises a heating system that is able to heat the link to cause the material to transition between the first state and the second state.

8. The system of claim 1, wherein a Young's modulus of the material when in the first state is lower than a Young's modulus of the material when in the second state.

9. The system of claim 1, wherein the temperature control system comprises a pipe through which a fluid can flow to change the temperature of the link.

10. The system of claim 1, wherein the temperature control system comprises an electrical heating element in the link.

11. A surgical system comprising:
a main tube;
a tip section at a distal end of the main tube, the tip section including a link comprising a shape memory alloy;
a tendon extending through the main tube and coupled to the tip section, wherein movement of the tendon causes actuation of the tip section; and
a heater coupled to the link, wherein the heater can be turned on to heat the shape memory alloy and cause the shape memory alloy to transition from a first state to a second state in which the link is stiff enough for actuation of the tip section using the tendon.

12. The system of claim 11, wherein the first state is a martensite state of the shape memory alloy, and the second state is an austenite state of the shape memory alloy.

13. The system of claim 11, wherein transition of the shape memory alloy from the first state to the second state stiffens the link for performing surgery with the surgical system.

14. The system of claim 11, wherein transition of the shape memory alloy from the first state to the second state removes bends made in the link during insertion of the instrument for a surgical procedure.

16. A surgical process, comprising:
inserting an entry guide in a patient;
inserting a tip section of an instrument through the entry guide, wherein during insertion of the tip section, a material in a link in the tip section is kept in a first state that provides the link with sufficient flexibility to bend while being inserted; and
changing a temperature of the link to cause the material in the link to transition to a second state after the link has reached a work site.

17. The process of claim 16, further comprising actuating the tip section using a tendon while the material is in the second state, wherein the material is more rigid in the second state than in the first state.

18. The process of claim 16, wherein the transition to the second state after the link has reached the work site, removes bends in the link formed when the tip section was inserted through the entry guide.

19. The process of claim 16, wherein the material comprises a shape memory alloy.

20. The process of claim 16, further comprising:
changing the temperature of the link to cause the material in the link to transition to the first state after the link has reached the work site; and
removing the tip section through the entry guide while the link is in the first state.
